(19) United States
(12) Patent Application Publication
(10) Pub. No.: US 2022/0347356 A1
(43) Pub. Date: Nov. 3, 2022

(54) Endovascular Prostheses

(71) Applicant: CorMatrix Cardiovascular, Inc., Roswell, GA (US)

(72) Inventor: Robert G Matheny, Norcross, GA (US)

(21) Appl. No.: 17/242,733

(22) Filed: Apr. 28, 2021

(51) Int. Cl. A61L 31/02; A61L 31/16; A61L 31/08

(52) U.S. Cl. CPC A61L 31/022; A61L 31/16; A61L 31/08; A61L 2400/16; A61L 2420/04; A61L 2300/414

(57) ABSTRACT

Low profile, self-expanding endovascular prostheses having a Ni-Ti alloy stent structure that is particularly well-suited for accessing and traversing narrow anatomical passageways and providing physiologically acceptable radial or hoop strength and longitudinal flexibility.

### FIELD OF THE INVENTION

The present invention relates generally to endoluminal devices designed for delivery into an anatomical passageway using minimally invasive techniques, such as percutaneous intravascular delivery. More particularly, the present invention relates to low profile, self-expanding endovascular prostheses having a stent structure that is particularly well-suited for accessing and traversing narrow anatomical passageways and providing physiologically acceptable radial or hoop strength and longitudinal flexibility.

### BACKGROUND OF THE INVENTION

As is well known in the art, endoluminal prostheses are generally tubular scaffolds that are configured and adapted to maintain narrow vascular and endoluminal ducts or tracts of the human body open and unoccluded. Conventional endoluminal prostheses generally fall within three classifications based on the stent structure or properties: balloon expandable, self-expanding and shape-memory.

Balloon expandable endoluminal prostheses are configured and adapted to transition from a compressed pre-deployment configuration, wherein the prostheses can be disposed in a vascular or endoluminal passageway, e.g., blood vessel, to an expanded post-deployment configuration, wherein the prostheses are positioned proximate the luminal wall of the passageway, by mechanical intervention, such as an expandable balloon catheter that is adapted to apply a radially outward positive pressure to mechanically deform the prostheses stent structure and, hence, prostheses to a larger diameter.

Self-expanding endoluminal prostheses are similarly configured and adapted to transition from a compressed pre-deployment configuration to an expanded post-deployment configuration. Self-expanding endoluminal prostheses are typically fabricated from stent materials that rebound when a positive pressure is exerted against the material, whereby a stent structure formed therewith transitions to a post-deployment configuration.

Self-expanding endoluminal prostheses are thus typically formed whereby the zero-stress configuration conforms to the post-deployment configuration, i.e., larger diameter. The self-expanding endoluminal prostheses are initially drawn down to the compressed pre-deployment configuration, i.e., smaller diameter, and constrained within a delivery catheter for endoluminal delivery. Removal of the constraint releases the constraining pressure and the self-expanding endoluminal prostheses, under its own mechanical properties, rebounds or transitions to the post-deployment configuration or larger diameter.

Finally, shape-memory endoluminal prostheses comprise stent structures that are fabricated from unique alloys that exhibit shape memory under certain thermal conditions. The stent structures of conventional shape-memory endoluminal prostheses typically comprise nickel-titanium alloys that are generally known as Nitinol®, which have a transition phase at or near normal body temperature, i.e., 37° C.

The prior art is replete with various endoluminal prosthesis stent designs across all endoluminal prostheses classifications.

There are, however, numerous drawbacks and disadvantages associated with conventional endoluminal prosthesis stent designs, which are, in many instances, due to the conflicting criteria between the desired radial or hoop strength and longitudinal or column strength and/or flexibility of the stent structure.

Typically, stent structures that are designed to optimize hoop strength typically sacrifice either column strength and/or longitudinal flexibility, while stent structures that are designed to optimize for column strength often compromise longitudinal flexibility and/or hoop strength.

In an effort to achieve a balance between hoop strength, column strength and longitudinal flexibility, many endoluminal stent structures employ a series of circumferential structural elements and longitudinal structural elements of varying configurations.

A large number of the noted stent structures utilize circumferential structural elements configured into a serpentine configuration or a zig-zag configuration. The reason underlying this configuration is the need for radial expansion of the stent.

Many of the stent structures that employ serpentine or zig-zag circumferential structural elements also employ longitudinal structural elements, which join adjacent circumferential structural elements and provide a modicum of longitudinal or column strength while retaining longitudinal flexibility of the device.

In addition to the complex stent structure, a further drawback associated with most conventional endoluminal prostheses, which, in many instances is due to the complex stent structure, is the size constraint of the endoluminal prostheses.

Indeed, heretofore, there are few, if any, endoluminal prostheses with a pre-deployment size, i.e., diameter, that is less than 10 μm, and no known endoluminal prostheses with a pre-deployment diameter less than 10 μm that provide physiologically acceptable radial or hoop strength and longitudinal flexibility.

There is thus a need to provide endoluminal prostheses with a pre-deployment diameter less than 10 μm that provide physiologically acceptable radial or hoop strength and longitudinal flexibility.

It is therefore an object of the present invention to provide endoluminal prostheses with a pre-deployment diameter less than 10 μm that provide physiologically acceptable radial or hoop strength and longitudinal flexibility.

It is another object of the present invention to provide endoluminal prostheses that induce remodeling of damaged cardiovascular tissue and regeneration of new cardiovascular tissue when disposed proximate the damaged tissue.

It is another object of the present invention to provide endoluminal prostheses that have the capacity to deliver biologically active agents, such as growth factors, and pharmacological agents, such as anti-inflammatories, to cardiovascular tissue, when disposed proximate thereto.

### SUMMARY OF THE INVENTION

The present invention is directed to low profile, self-expanding endovascular prostheses that can be readily employed to treat various anomalies in anatomical passageways; particularly, abnormalities in blood vessels, such as vascular saccular and fusiform aneurysms, and to maintain patency of blood vessels after angioplasty.

In one preferred embodiment of the invention, the endovascular prostheses comprise a single linear wire stent structure.

In a preferred embodiment, the wire stent structure comprises a nickel-titanium (Ni—Ti) alloy (referred to hereinafter as Nitinol®).

In a preferred embodiment of the invention, the Nitinol® wire stent structure comprises a superelastic structure, which is adapted to transition from a pre-deployment configuration, wherein the stent structure can be disposed in a delivery catheter, to an expanded post-deployment coil configuration when subjected to a pre-defined temperature, i.e., martensite-austinite transition temperature (A_{f}).

In a preferred embodiment, when the Nitinol® wire stent structure is in the expanded post-deployment configuration (or state), the Nitinol® wire stent structure comprises a coil shape, i.e., a plurality of windings.

In a preferred embodiment, the wire stent structure further comprises an outer coating.

In some embodiments of the invention, the outer coating comprises an immunomodulating compound.

In some embodiments, the outer coating comprises an extracellular matrix (ECM) composition comprising acellular ECM derived from a mammalian tissue source.

In some embodiments, the outer coating comprises an ECM-mimicking composition comprising poly(glycerol sebacate) (PGS).

In some embodiments, the outer coating comprises an ECM/ECM-mimicking composition comprising acellular ECM and PGS.

In some embodiments, the ECM composition and/or ECM-mimicking composition and/or ECM/ECM-mimicking composition further comprises at least one additional biologically active agent or composition, i.e., an agent that induces or modulates a physiological or biological process, or cellular activity, e.g., induces proliferation, and/or growth and/or regeneration of tissue.

In some embodiments, the biologically active agent comprises a growth factor, such as, without limitation, a transforming growth factor-alpha (TGF-α), transforming growth factor-beta (TGF-β), basic fibroblast growth factor (bFGF) and vascular endothelial growth factor (VEGF).

In some embodiments, the ECM composition and/or ECM-mimicking composition and/or ECM/ECM-mimicking composition further comprises at least one pharmacological agent or composition (or drug), i.e., an agent or composition that is capable of producing a desired biological effect in vivo, e.g., stimulation or suppression of apoptosis, stimulation or suppression of an immune response, etc.

Suitable pharmacological agents and compositions include, without limitation, antibiotics, anti-fibrotics, anti-viral agents, analgesics, anti-inflammatories, anti-neoplastics, anti-spasmodics, and anti-coagulants and/or anti-thrombotic agents.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIGS. 1A-1C are schematic illustrations of arterial aneurysms;

FIG. 2A is a side plan view of one embodiment of an endovascular prosthesis, i.e., a wire stent structure, in a pre-deployment configuration, in accordance with the invention;

FIG. 2B is a side plan view of the distal end of the endovascular prosthesis shown in FIG. 2A in an expanded post-deployment configuration, in accordance with the invention;

FIG. 2C is a side plan view of the endovascular prosthesis shown in FIG. 2B disposed in a delivery catheter, in accordance with the invention;

FIG. 3A is a side plan view of another embodiment of an endovascular prosthesis disposed in a delivery catheter, having the stent structure shown in FIG. 2B and an outer covering or graft, in accordance with the invention;

FIG. 3B is a side plan view of the endovascular prosthesis shown in FIG. 3A in an expanded post-deployment configuration, in accordance with the invention;

FIG. 4A is a side plan view of one embodiment of a hybrid endovascular prosthesis in a pre-formed configuration, in accordance with the invention;

FIG. 4B is a side plan view of the hybrid endovascular prosthesis shown in FIG. 4A with a deformable region and a formed superelastic coil shaped end region, in accordance with the invention;

FIG. 4C is a side plan view of the hybrid endovascular prosthesis shown in FIG. 4B with the superelastic end region in a post-forming linear configuration, in accordance with the invention;

FIG. 4D is a further side plan view of the hybrid endovascular prosthesis shown in FIG. 4B with an expanded view of one embodiment of a work hardened, high-stress region, in accordance with the invention;

FIG. 4E is a side plan view of the hybrid endovascular prosthesis shown in FIG. 4D in a post-deployment configuration, in accordance with the invention;

FIG. 4F is a side plan view of the hybrid endovascular prosthesis shown in FIG. 4E showing the superelastic end region severed from the deformable region at the work hardened, high-stress region, in accordance with the invention;

FIG. 5A is a side plan view of another embodiment of a hybrid endovascular prosthesis shown in FIG. 4C having an outer covering or graft disposed superelastic end region, in accordance with the invention;

FIG. 5B is a side plan view of the hybrid endovascular prosthesis shown in FIG. 5A showing the superelastic end region in a post-deployment configuration and severed from the deformable region at the work hardened, high-stress region, in accordance with the invention;

FIG. 6A is a side plan view of the endovascular prosthesis shown in FIG. 2B disposed in a blood vessel proximate an aneurysm, in accordance with the invention;

FIG. 6B is a side plan view of the superelastic region of the endovascular prosthesis shown in FIG. 4F disposed in a blood vessel proximate an aneurysm, in accordance with the invention;

FIG. 7A is a side plan view of the endovascular prosthesis shown in FIG. 3B disposed in a blood vessel proximate an aneurysm, in accordance with the invention; and

FIG. 7B is a side plan view of the superelastic region of the endovascular prosthesis shown in FIG. 5B disposed in a blood vessel proximate an aneurysm, in accordance with the invention.

### DETAILED DESCRIPTION OF THE PREFERRED EMBODIMENT

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred apparatus, systems, structures and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein are hereby incorporated by reference herein in their entirety.

As used in this specification and the appended claims, the singular forms “a”, “an” and “the” include plural referents unless the content clearly dictates otherwise. Thus, for example, reference to “a pharmacological agent” includes two or more such agents and the like.

Further, ranges can be expressed herein as from “about” or “approximately” one particular value, and/or to “about” or “approximately” another particular value. When such a range is expressed, another embodiment includes from the one particular value and/or to the other particular value. Similarly, when values are expressed as approximations, by use of the antecedent “about” or “approximately”, it will be understood that the particular value foams another embodiment. It will be further understood that the endpoints of each of the ranges are significant both in relation to the other endpoint, and independently of the other endpoint.

It is also understood that there are a number of values disclosed herein, and that each value is also herein disclosed as “about” or “approximately” that particular value in addition to the value itself. For example, if the value “10” is disclosed, then “approximately 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed then “less than or equal to 10” as well as “greater than or equal to 10” is also disclosed.

### Definitions

The term “endovascular prosthesis”, as used herein, means and includes a device designed, configured and/or adapted to be positioned in an anatomical passageway, such as a blood vessel, to treat an anomaly associated therewith and/or keep the passageway open and unoccluded. The term “endovascular prosthesis” thus includes, without limitation, vascular stents, grafts and vaso-occlusive devices.

The terms “superelastic” and “pseudoelastic” are used interchangeably herein, and mean and include a shape-memory alloy (SMA) that has the inherent capability to undergo deformation at one temperature, stay in the deformed configuration when the force(s) exerted to deform the SMA has been removed, then recover the original deformed configuration upon heating the SMA above a transformation temperature.

The terms “superelastic” and “pseudoelastic” as used herein in connection with Ni-Ti alloys; particularly, Nitinol® alloys, thus mean and include a Nitinol® alloy that has the inherent capability to undergo a crystal phase transformation from a martensite crystal structure to an austenite crystal structure at a pre-defined transformation temperature and be deformed at or above the transformation temperature, stay in the deformed configuration when the force(s) exerted to deform the SMA has been removed, transition from the austenite crystal structure back to the martensite crystal structure when the Nitinol® alloy is cooled below the transformation temperature, and then revert back to the original deformed configuration upon heating the Nitinol® alloy above a transformation temperature.

The terms “extracellular matrix”, “ECM”, and “ECM material” are used interchangeably herein, and mean and include a collagen-rich substance that is found in between cells in mammalian tissue, and any material processed therefrom, e.g., decellularized ECM.

The term “acellular ECM”, as used herein, means ECM that has a reduced content of cells.

According to the invention, ECM can be derived from a variety of mammalian tissue sources and tissue derived therefrom, including, without limitation, small intestine submucosa (SIS), urinary bladder submucosa (UBS), stomach submucosa (SS), central nervous system tissue, epithelium of mesodermal origin, i.e., mesothelial tissue, dermal tissue, subcutaneous tissue, gastrointestinal tissue, tissue surrounding growing bone, placental tissue, omentum tissue, cardiac tissue, kidney tissue, pancreas tissue, lung tissue, and combinations thereof. The ECM can also comprise collagen from mammalian sources.

The terms “heart tissue” and “cardiac tissue” are used collectively herein, and mean and include, without limitation, mammalian tissue derived from any cardiovascular structure including, without limitation, pericardial tissue, myocardial tissue, vascular tissue and the like.

The terms “mammalian-based tissue”, “collagenous mammalian tissue” and “collagenous tissue” are used collectively herein, and mean and include, without limitation, tissue that is also derived from a mammalian tissue source.

According to the invention, the mammalian-based tissue and collagenous mammalian tissue can similarly be derived from a variety of mammalian tissue sources and tissue derived therefrom, including, without limitation, the heart, small intestine, large intestine, stomach, lung, liver, kidney, pancreas, peritoneum, placenta, amniotic membrane, umbilical cord, bladder, prostate, and any fetal tissue from any mammalian organ.

The mammalian-based tissue and collagenous mammalian tissue can also be derived from a mammalian tissue source that is devoid of xenogeneic antigens, including, without limitation, collagenous mammalian tissue that is devoid of one of the following xenogeneic antigens: galactose-alpha-1,3-galactose (also referred to as α-gal), beta-1,4 N-acetylgalactosaminyltransferase 2, membrane cofactor protein, hepatic lectin H1, cytidine monophospho-N-acetylneuraminic acid hydroxylase, swine leukocyte antigen class I and porcine endogenous retrovirus polymerase (referred to herein as “immune privileged collagenous mammalian tissue”).

The term “genetically modified organism”, as used herein means and includes any living organism that has at least one gene modified by artificial means, e.g., gene editing.

The term “immune privileged collagenous mammalian tissue”, as used herein means and includes xenogeneic collagenous mammalian tissue that can be disposed proximate mammalian tissue with a minimal or virtually absent adverse immune response; particularly, an adverse immune response associated with xenogeneic tissue graft rejection.

According to the invention, the term “mammalian” means and includes, without limitation, warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The term “crosslinked collagenous mammalian tissue”, as used herein, means and includes mammalian tissue that exhibits at least 25% chemical bonding of adjacent chains of molecules, i.e., collagen fibrils, which comprise the collagenous mammalian tissue.

The term “polymer”, as used herein means and includes, without limitation, polyurethane urea, porous polyurethane urea (Artelon®), polypropylene, poly(ϵ-caprolactone) (PCL), poly(glycerol sebacate) (PGS), polytetrafluoroethylene (PTFE), poly(styrene-block-isobutylene-block-Styrene) (SIBS), polyglycolide (PGA), polylactide (PLA), polydioxanone (a polyether-ester), polylactide-co-glycolide, polyamide esters, polyalkalene esters, polyvinyl esters, polyvinyl alcohol, polyanhydrides, polyurethanes, polydimethylsiloxanes, poly(ethylene glycol), polytetrafluoroethylene (Teflon™) and polyethylene terephthalate (Dacron™)

The term “biologically active agent”, as used herein, means and includes an agent that induces or modulates a physiological or biological process, or cellular activity, e.g., induces proliferation, and/or growth and/or regeneration of tissue.

The term “biologically active agent” thus means and includes a growth factor, including, without limitation, fibroblast growth factor-2 (FGF-2), transforming growth factor beta (TGF-β) and vascular endothelial growth factor (VEGF).

The term “biologically active agent” also means and includes a cell, including, without limitation, human embryonic stem cells, myofibroblasts, mesenchymal stem cells, and hematopoietic stem cells.

The term “biologically active agent” also means and includes an exosome and/or microsome.

The terms “exosome” and “microsome” as used herein mean and include a lipid bilayer structure that contains or encapsulates a biologically active agent and/or pharmacological agent, including, without limitation, a growth factor, e.g., TGF-β, TGF-α, VEGF and insulin-like growth factor (IGF-I), a cytokine, e.g., interleukin-10 (IL-10), a transcription factor and microRNA (miRNA).

The term “biologically active agent” also means and includes agents commonly referred to as a “protein”, “peptide” and “polypeptide”, including, without limitation, collagen (types I-V), proteoglycans and glycosaminoglycans (GAGs).

The terms “pharmacological agent”, “active agent” and “drug” are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect or effects in animals, including warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The terms “pharmacological agent”, “active agent” and “drug” thus mean and include, without limitation, antibiotics, anti-arrhythmic agents, anti-viral agents, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPs), enzymes and enzyme inhibitors, anticoagulants and/or antithrombotic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

The terms “pharmacological agent”, “active agent” and “drug” also mean and include, without limitation, atropine, tropicamide, dexamethasone, dexamethasone phosphate, betamethasone, betamethasone phosphate, prednisolone, triamcinolone, triamcinolone acetonide, fluocinolone acetonide, anecortave acetate, budesonide, cyclosporine, FK-506, rapamycin, ruboxistaurin, midostaurin, flurbiprofen, suprofen, ketoprofen, diclofenac, ketorolac, nepafenac, lidocaine, neomycin, polymyxin b, bacitracin, gramicidin, gentamicin, oyxtetracycline, ciprofloxacin, ofloxacin, tobramycin, amikacin, vancomycin, cefazolin, ticarcillin, chloramphenicol, miconazole, itraconazole, trifluridine, vidarabine, ganciclovir, acyclovir, cidofovir, ara-amp, foscarnet, idoxuridine, adefovir dipivoxil, methotrexate, carboplatin, phenylephrine, epinephrine, dipivefrin, timolol, 6-hydroxydopamine, betaxolol, pilocarpine, carbachol, physostigmine, demecarium, dorzolamide, brinzolamide, latanoprost, sodium hyaluronate, insulin, verteporfin, pegaptanib, ranibizumab, and other antibodies, antineoplastics, anti-VEGFs, ciliary neurotrophic factor, brain-derived neurotrophic factor, bFGF, Caspase-1 inhibitors, Caspase-3 inhibitors, α-Adrenoceptors agonists, NMDA antagonists, Glial cell line-derived neurotrophic factors (GDNF), pigment epithelium-derived factor (PEDF), NT-3,NT-4, NGF and IGF-2.

The terms “pharmacological agent”, “active agent” and “drug” also mean and include the Class I-Class V antiarrhythmic agents disclosed in Applicant's U.S. Pat Nos. 9,119,841, 10,188,509, 10,188,510, 10,143,778 and 10,952,843, and Co-pending App. No. 16/990,236, including, without limitation, (Class Ia) quinidine, procainamide and disopyramide; (Class Ib) lidocaine, phenytoin and mexiletine; (Class Ic) flecainide, propafenone and moricizine; (Class II) propranolol, esmolol, timolol, metoprolol and atenolol; (Class III) amiodarone, sotalol, ibutilide and dofetilide; (Class IV) verapamil and diltiazem) and (Class V) adenosine and digoxin.

The terms “pharmacological agent”, “active agent” and “drug” also mean and include, without limitation, the antibiotics disclosed in Applicant's U.S. Pat. Nos. 9,119,841, 10,188,509, 10,188,510, 10,143,778 and 10,952,843, and Co-pending App. No. 16/990,236, including, without limitation, aminoglycosides, cephalosporins, chloramphenicol, clindamycin, erythromycins, fluoroquinolones, macrolides, azolides, metronidazole, penicillin, tetracyclines, trimethoprim-sulfamethoxazole, gentamicin and vancomycin.

As indicated above, the terms “pharmacological agent”, “active agent” and “drug” also mean and include an anti-inflammatory.

The terms “anti-inflammatory” and “anti-inflammatory agent” are also used interchangeably herein, and mean and include a “pharmacological agent” and/or “active agent formulation”, which, when a therapeutically effective amount is administered to a subject, prevents or treats bodily tissue inflammation i.e., the protective tissue response to injury or destruction of tissues, which serves to destroy, dilute, or wall off both the injurious agent and the injured tissues.

The terms “anti-inflammatory” and “anti-inflammatory agent” thus include the anti-inflammatories disclosed in Applicant's U.S. Pat. Nos. 9,119,841, 10,188,509, 10,188,510, 10,143,778 and 10,952,843, and Co-pending App. No. 16/990,236, including, without limitation, desoximetasone, dexamethasone dipropionate, cloticasone propionate, diftalone, fluorometholone acetate, fluquazone, meseclazone, mesterolone, methandrostenolone, methenolone, methenolone acetate, methylprednisolone suleptanate, halopredone acetate, alclometasone dipropionate, apazone, balsalazide disodium, cintazone cormethasone acetate, cortodoxone, diflorasone diacetate, diflumidone sodium, endrysone, fenpipalone, flazalone, fluretofen, fluticasone propionate, isoflupredone acetate, nabumetone, nandrolone, nimazone, oxyphenbutazone, oxymetholone, phenbutazone, pirfenidone, prifelone, proquazone, rimexolone, seclazone, tebufelone and testosterone.

The terms “pharmacological agent”, “active agent” and “drug” also mean and include the statins, i.e., HMG-CoA reductase inhibitors, disclosed in Applicant's U.S. Pat. Nos. 9,119,841, 10,188,509, 10,188,510, 10,143,778 and 10,952,843, and Co-pending App. No. 16/990,236, including, without limitation, atorvastatin, cerivastatin, fluvastatin and lovastatin.

The terms “pharmacological agent”, “active agent”, “drug” and “active agent formulation” further mean and include the anti-proliferative agents disclosed in Applicant's U.S. Pat Nos. 9,119,841, 10,188,509, 10,188,510, 10,143,778and 10,952,843, and Co-pending App. No. 16/990,236, including, without limitation, paclitaxel, sirolimus and derivatives thereof, including everolimus.

The term “pharmacological composition”, as used herein, means and includes a composition comprising a “pharmacological agent” and/or any additional agent or component identified herein.

Additional biologically active and pharmacological agents are set forth in priority U.S. application Ser. No. 15/206,833, now U.S. Pat. No. 10,188,510, which is expressly incorporated herein in its entirety.

The term “therapeutically effective”, as used herein, means that the amount of the “pharmacological agent” and/or “biologically active agent” and/or “pharmacological composition” and/or “biologically active composition” administered is of sufficient quantity to ameliorate one or more causes, symptoms, or sequelae of a disease or disorder. Such amelioration only requires a reduction or alteration, not necessarily elimination, of the cause, symptom, or sequelae of a disease or disorder.

The terms “patient” and “subject” are used interchangeably herein, arid mean and include warm blooded mammals, humans and primates; avians; domestic household or faun animals, such as cats, dogs, sheep, goats, cattle, horses and pigs; laboratory animals, such as mice, rats and guinea pigs; fish; reptiles; zoo and wild animals; and the like.

The term “comprise” and variations of the term, such as “comprising” and “comprises,” means “including, but not limited to” and is not intended to exclude, for example, other additives, components, integers or steps.

The term “comprise” and variations of the term, such as “comprising” and “comprises,” as used in connection with a prosthetic valve composition and/or mammalian tissue, also means a composition and/or mammalian tissue employed to form a prosthetic valve structure, such as a sheet member, and, hence, a prosthetic valve of the invention.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

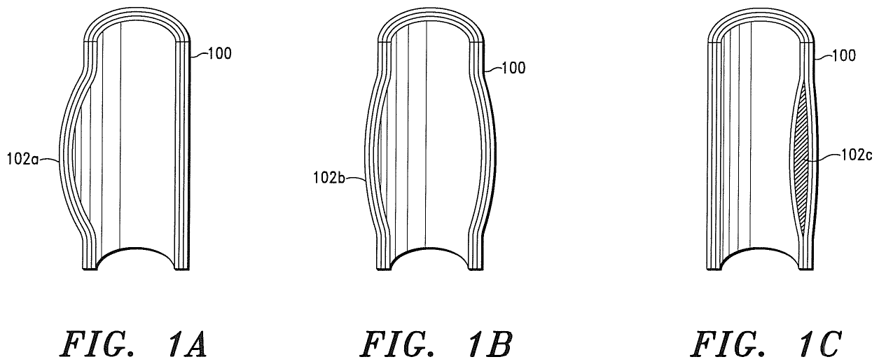

As stated above, the present invention is directed to endovascular prostheses that can be readily employed to treat various anomalies in anatomical passageways; particularly, abnormalities in blood vessels, such as the vascular saccular and fusiform aneurysms shown in vessel 100 in Figs. 1A and 1B, denoted 102a and 102b, respectively, and vascular dissections, such as the vascular dissection 102c in vessel 100 shown in FIG. 1C, and to maintain patency of blood vessels after angioplasty.

In some embodiments of the invention, the endovascular prostheses are configured and adapted to readily access and translate through anatomical passageways with lumen diameters less than 1 mm.

In some embodiments of the invention, the endovascular prostheses and configured and adapted to readily access and translate through anatomical passageways with lumen diameters less than 10 μm.

As also stated above, in a preferred embodiment of the invention, the endovascular prostheses comprise a single linear wire stent structure that is particularly well-suited for accessing and traversing narrow anatomical passageways and providing physiologically acceptable radial or hoop strength and longitudinal flexibility.

According to the invention, the wire stent structure can comprise various biocompatible metals and alloys, including stainless steel and alloys comprising same, magnesium and alloys comprising same, and shape memory alloys, including, without limitation, nickel-titanium (Ni-Ti) alloys.

As discussed in detail below, in a preferred embodiment, the wire stent structure comprises a Ni-Ti alloy (referred to hereinafter as Nitinol®).

It is, however, understood that, although the exemplary embodiments of the endovascular prostheses of the invention are described and illustrated herein in connection with wire stent structures comprising Ni-Ti alloys, and specifically Nitinol®, the invention is not limited to wire stent structures comprising Ni-Ti alloys. Indeed, the teachings disclosed herein can also be readily employed to provide wire stent structures of the invention comprising other shape-memory alloys, such as, by way of example, Fe-Mn-Si, Cu-Zn-Al and Cu-Al-Ni alloys.

In a preferred embodiment of the invention, the Nitinol® wire stent structure comprises a superelastic structure, which is adapted to transition from a pre-deployment configuration, wherein the stent structure can be disposed in a delivery catheter, to an expanded post-deployment coil configuration when subjected to a pre-defined temperature, i.e., martensite-austinite transition temperature (A_{f}).

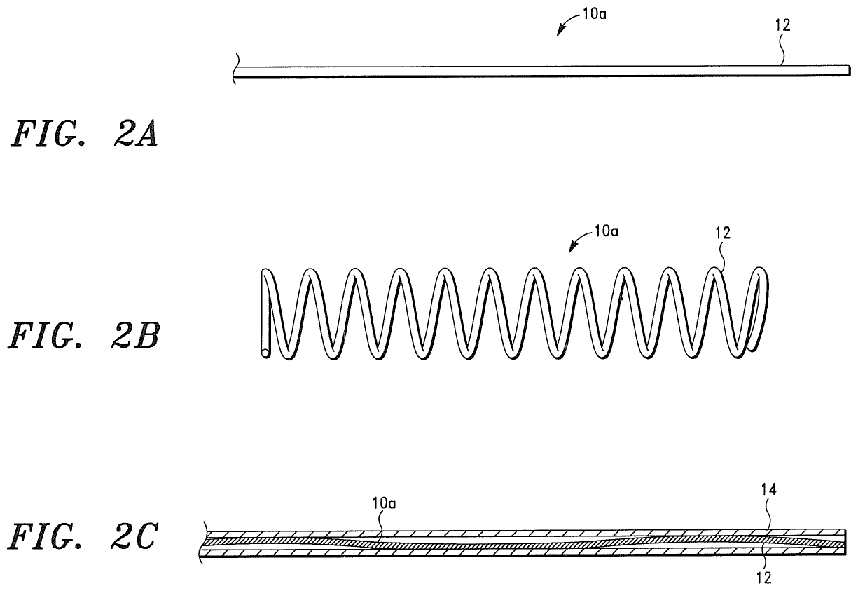

In a preferred embodiment, when the Nitinol® wire stent structure is in the expanded post-deployment configuration (or state), the Nitinol® wire stent structure comprises a coil shape, i.e., a plurality of windings, as shown in FIG. 2B.

According to the invention, the Nitinol® wire stent structure, when in the expanded post-deployment configuration (or state), can comprise various lengths.

In a preferred embodiment, the Nitinol® wire stent structure, when in the expanded post-deployment configuration (or state), comprises a length in the range of approximately 6.0 mm to 100.0 mm.

According to the invention, the Nitinol® wire stent structure, when in the expanded post-deployment configuration (or state), can further comprise various diameters.

In a preferred embodiment, the Nitinol® wire stent structure, when in the expanded post-deployment configuration (or state), comprises a diameter in the range of approximately 10.0 μm to 25.0 mm.

In some embodiments of the invention, the Nitinol® wire stent structure comprises multiple physical property regions, which are generated via differential thermal treatments and mechanical processing.

As discussed in detail below, in a preferred embodiment, the multiple physical property regions include (i) a superelastic region disposed on the distal end of the stent structure, which, as discussed in detail below, is similarly adapted to transition from a pre-deployment substantially linear configuration to a post-deployment coil configuration when subjected to a pre-defined temperature, i.e., A_{f}, (ii) a deformable proximal region or end and (iii) a mechanically deformed, i.e., work hardened, high stress retention region therebetween.

The combination of physical property regions provides unique characteristics that allow good trackability of the stent structure and, hence, endovascular prosthesis formed therewith, (i.e., ease of maneuvering through anatomical passageways), good flexibility, a low profile, good conformability and high radial force when deployed, and, as discussed in detail below, the capability of fracturing and releasing the superelastic physical property region from the deformable region when subjected to a pre-defined A_{f} temperature.

According to the invention, the superelastic region of the Nitinol® wire stent structure, when in the expanded post-deployment configuration (or state), can similarly comprise various lengths.

In a preferred embodiment, the superelastic region of the Nitinol® wire stent structure, when in the expanded post-deployment configuration (or state), similarly comprises a length in the range of approximately 6.0 mm to 100.0 mm.

According to the invention, the superelastic region of the Nitinol® wire stent structure, when in the expanded post-deployment configuration (or state), can similarly comprise various diameters.

In a preferred embodiment, the superelastic region of the Nitinol® wire stent structure, when in the expanded post-deployment configuration (or state), comprises a diameter in the range of approximately 5.0 μm to 10.0 μm.

In a preferred embodiment, the wire stent structures of the invention; particularly, the Nitinol® wire stent structures, further comprise an outer coating.

In some embodiments of the invention, the outer coating comprises an immunomodulating compound.

In some embodiments, the immunomodulating compound comprises a polysaccharide, including, without limitation, GAGs, dextrans, alginate and chitosan.

In some embodiments, the immunomodulating compound comprises a polymeric material, including, without limitation, high molecular weight hyaluronic acid (HMW-HA).

In some embodiments of the invention, the outer coating comprises an extracellular matrix (ECM) composition comprising acellular ECM derived from a mammalian tissue source.

In a preferred embodiment of the invention, the mammalian tissue source is selected from the group comprising small intestine submucosa (SIS), urinary bladder submucosa (UBS), stomach submucosa (SS), central nervous system tissue, mesodermal tissue, i.e. mesothelial tissue, dermal extracellular matrix, subcutaneous extracellular matrix, gastrointestinal extracellular matrix, i.e., large and small intestines, tissue surrounding growing bone, placental extracellular matrix, omentum extracellular matrix, cardiac extracellular matrix, e.g., pericardium and/or myocardium, kidney extracellular matrix, pancreas extracellular matrix, lung extracellular matrix, and combinations thereof.

In some embodiments of the invention, the ECM composition further comprises at least one additional biologically active agent or composition, i.e., an agent that induces or modulates a physiological or biological process, or cellular activity, e.g., induces proliferation, and/or growth and/or regeneration of tissue.

In some embodiments, the biologically active agent comprises a growth factor, such as, without limitation, a transforming growth factor-alpha (TGF-α), transforming growth factor-beta (TGF-β), basic fibroblast growth factor (bFGF) and vascular endothelial growth factor (VEGF).

In some embodiments, the ECM composition further comprises at least one pharmacological agent or composition (or drug), i.e., an agent or composition that is capable of producing a desired biological effect in vivo, e.g., stimulation or suppression of apoptosis, stimulation or suppression of an immune response, etc.

Suitable pharmacological agents and compositions include, without limitation, antibiotics, anti-fibrotics, anti-viral agents, analgesics, anti-inflammatories, anti-neoplastics, anti-spasmodics, and anti-coagulants and/or anti-thrombotic agents.

In some embodiments of the invention, the pharmacological agent comprises an antibiotic, such as, without limitation, vancomycin and gentamicin.

In some embodiments of the invention, the pharmacological agent comprises an antimicrobial, such as, without limitation, silver particles and copper particles.

In a preferred embodiment of the invention, the outer coating comprises an ECM-mimicking composition comprising poly(glycerol sebacate) (PGS).

In some embodiments, the outer coating comprises an ECM/ECM-mimicking composition comprising acellular ECM derived from one of the aforementioned mammalian tissue sources and PGS.

In some embodiments, the ECM and/or ECM/ECM-mimicking composition further comprises at least one of the aforementioned biologically active agents and/or pharmacological agents.

In some embodiments of the invention, the outer coating comprises a thermal insulating composition comprising at least one thermal insulating material.

In a preferred embodiment, the thermal insulating composition comprises a biodegradable thermal insulating composition comprising at least one biodegradable thermal insulating material.

According to the invention, suitable thermal insulating materials can comprise polymers having a thermal conductivity less than 0.25 W/(m*K); particularly, polylactide (PLA).

In some embodiments of the invention, the endovascular prostheses further comprise an outer covering or graft, such as disclosed in Applicant's U.S. Pat. Nos. 9,533,072and 10,188,509, which are incorporated by reference herein.

In a preferred embodiment, the graft comprises a seamless structure.

Preferably, the graft is similarly adapted to transition from a pre-deployment configuration to a post-deployment configuration.

According to the invention, the graft can similarly comprise various biocompatible materials.

In some embodiments of the invention, the graft comprises a polymeric composition comprising a biodegradable polymeric material.

According to the invention, suitable biodegradable polymeric materials comprise, without limitation, polycaprolactone (PCL), Artelon® (porous polyurethaneurea), polyglycolide (PGA), polylactide (PLA), poly(ϵ-caprolactone) (PCL), poly dioxanone (a polyether-ester), poly lactide-co-glycolide, polyamide esters, polyalkalene esters, polyvinyl esters, polyvinyl alcohol, and polyanhydrides.

According to the invention, the polymeric composition can further comprise a hydrogel composition, including, without limitation, polyurethane, poly(ethylene glycol), poly(propylene glycol), poly(vinylpyrrolidone), xanthan, methyl cellulose, carboxymethyl cellulose, alginate, hyaluronan, poly(acrylic acid), polyvinyl alcohol, acrylic acid, hydroxypropyl methyl cellulose, methacrylic acid, αβ-glycerophosphate, κ-carrageenan, 2-acrylamido-2-methylpropanesulfonic acid, and β-hairpin peptide.

According to the invention, the polymeric composition can further comprise a non-biodegradable polymer, including, without limitation, polytetrafluoroethylene (Teflon®) and polyethylene terephthalate (Dacron®).

In some embodiments of the invention, the polymeric composition comprises poly(urethane urea); preferably, Artelon® distributed by Artimplant AB in Goteborg, Sweden.

In some embodiments of the invention, the graft comprises an immune privileged collagenous mammalian tissue, as defined herein.

In some embodiments of the invention, the graft comprises an ECM composition, such as described above.

In some embodiments, the immune privileged collagenous mammalian tissue and/or polymeric composition and/or ECM composition and, hence, graft comprising same, further comprises at least one of the aforementioned biologically active agents and/or pharmacological agents.

In some embodiments of the invention, it is thus contemplated that, following placement of an endovascular prosthesis of the invention in an anatomical passageway, such as a blood vessel, and, hence, tissue associated therewith, the endovascular prosthesis will induce “modulated healing” of the anatomical passageway and tissue associated therewith.

The term “modulated healing”, as used herein, and variants of this language generally refer to the modulation (e.g., alteration, delay, retardation, reduction, etc.) of a process involving different cascades or sequences of naturally occurring tissue repair in response to localized tissue damage or injury, substantially reducing their inflammatory effect.

Modulated healing, as used herein, includes many different biologic processes, including epithelial growth, fibrin deposition, platelet activation and attachment, inhibition, proliferation and/or differentiation, connective fibrous tissue production and function, angiogenesis, and several stages of acute and/or chronic inflammation, and their interplay with each other.

For example, in some embodiments of the invention, an endovascular prosthesis of the invention is specifically formulated (or designed) to alter, delay, retard, reduce, and/or detain one or more of the phases associated with healing of damaged tissue, including, but not limited to, the inflammatory phase (e.g., platelet or fibrin deposition), and the proliferative phase when in contact with biological tissue.

In some embodiments, “modulated healing” means and includes the ability of an endovascular prosthesis of the invention to restrict the expression of inflammatory components.

By way of example, according to the invention, when an endovascular prosthesis of the invention comprises a statin and the endovascular prosthesis is positioned proximate damaged tissue, the endovascular prosthesis will restrict expression of monocyte chemoattractant protein-1 (MCP-1) and chemokine (C-C) motif ligand 2 (CCR2).

By way of further example, according to the invention, when an endovascular prosthesis comprises an immune privileged collagenous mammalian tissue, as defined herein, and the endovascular prosthesis is positioned proximate damaged tissue, the endovascular prosthesis will not induce an adverse immune response; particularly, an immune response associated with tissue prosthesis rejection in vivo.

In some embodiments of the invention, “modulated healing” means and includes the ability of an endovascular prosthesis of the invention to alter a substantial inflammatory phase (e.g., platelet or fibrin deposition) at the beginning of the tissue healing process. As used herein, the phrase “alter a substantial inflammatory phase” refers to the ability of an endovascular prosthesis of the invention to substantially reduce the inflammatory response at a damaged tissue site, e.g., cardiovascular vessel, when in contact with tissue at the site.

The term “modulated healing” also refers to the ability of an endovascular prosthesis of the invention to induce cell migration, and cell and host tissue proliferation when disposed proximate damaged tissue.

The term “modulated healing” also refers to the ability of an endovascular prosthesis of the invention to induce host tissue proliferation, bioremodeling, including neovascularization, e.g., vasculogenesis, angiogenesis, and intussusception, and regeneration of new tissue and tissue structures with site-specific structural and functional properties, when disposed proximate damaged tissue.

Thus, in some embodiments of the invention, the term “modulated healing” means and includes the ability of an endovascular prosthesis of the invention to modulate inflammation and induce host tissue proliferation and remodeling, and regeneration of new tissue when disposed proximate damaged tissue.

Referring now to FIGS. 2A and 2B, there is shown one embodiment of an endovascular prosthesis of the invention.

As illustrated in FIG. 2A, in a preferred embodiment of the invention, the endovascular prosthesis 10a comprises a single linear wire stent structure 12.

As indicated above, in a preferred embodiment, the wire stent structure comprises a Ni-Ti alloy, more preferably, Nitinol®.

As also indicated above, in a preferred embodiment of the invention, the Nitinol® wire stent structure comprises a superelastic structure, which is adapted to transition from a pre-deployment configuration, such as shown in FIG. 2A, wherein the stent structure 12 can be disposed in a delivery catheter, such as delivery catheter 14 shown in FIG. 2C, to an expanded post-deployment coil configuration, such as shown in FIG. 2B, when subjected to a pre-defined temperature, i.e., martensite-austinite transition temperature (A_{f}).

As illustrated in FIG. 2B, in a preferred embodiment, when the Nitinol® wire stent structure 12 is in the expanded post-deployment configuration (or state), the Nitinol® wire stent structure 12 comprises a coil shape, i.e., a plurality of windings.

According to the invention, the superelastic structure is provided via standard Ni-Ti alloy processing known in the art.

In a preferred embodiment, when the Nitinol® wire stent structure 12 is in the expanded post-deployment configuration (or state), the Nitinol® wire stent structure 12 comprises optimal radial or hoop strength and flexibility.

As indicated above, in a preferred embodiment, the Nitinol® wire stent structure 12 further comprises an outer coating.

In some embodiments of the invention, the outer coating comprises one of the aforementioned immunomodulating compounds.

In some embodiments, the outer coating comprises one of the aforementioned ECM compositions.

In some embodiments, the outer coating comprises one of the aforementioned polymeric compositions.

In some embodiments, the outer coating comprises one of the aforementioned ECM-mimicking compositions comprising poly(glycerol sebacate) (PGS).

In some embodiments of the invention, the outer coating comprises one of the aforementioned ECM/ECM-mimicking compositions comprising acellular ECM derived from one of the aforementioned mammalian tissue sources and PGS.

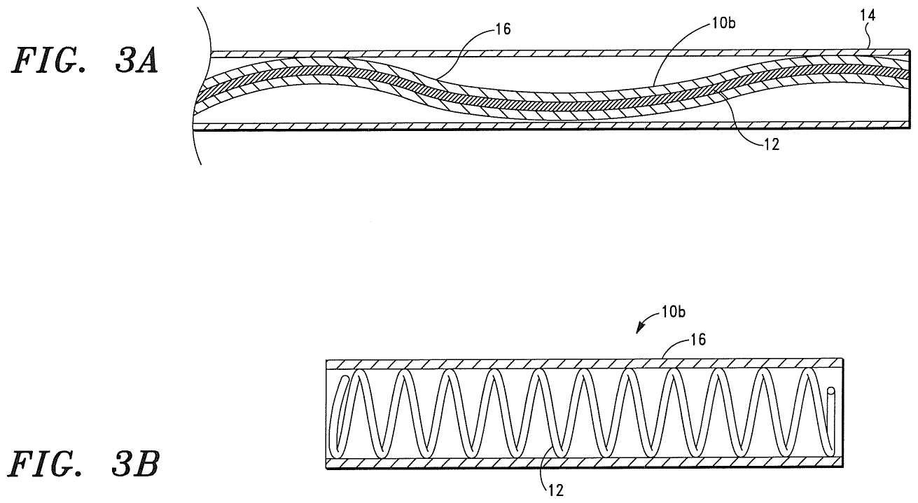

Referring now to FIGS. 3A and 3B, there is shown another embodiment of an endovascular prosthesis of the invention, denoted 10b, disposed in the delivery catheter 14 shown in FIG. 2C.

As illustrated in FIGS. 3A and 3B, the endovascular prosthesis 10 b comprises the same Nitinol® wire stent structure 12 shown in FIG. 2C. However, in this embodiment, the endovascular prosthesis 10b further comprises an outer covering or graft 16.

In a preferred embodiment of the invention, the graft 16 is also adapted to transition from a pre-deployment configuration to a post-deployment configuration, as shown in FIG. 3B.

As indicated above, according to the invention, the graft 16 can comprise various biocompatible materials.

In some embodiments of the invention, the graft 16 comprises one of the aforementioned polymeric compositions.

In some embodiments, the graft 16 comprises an immune privileged collagenous mammalian tissue.

In some embodiments, the graft 16 comprises one of the aforementioned ECM/ECM-mimicking compositions.

In a preferred embodiment of the invention, the graft 16 comprises one of the aforementioned ECM compositions.

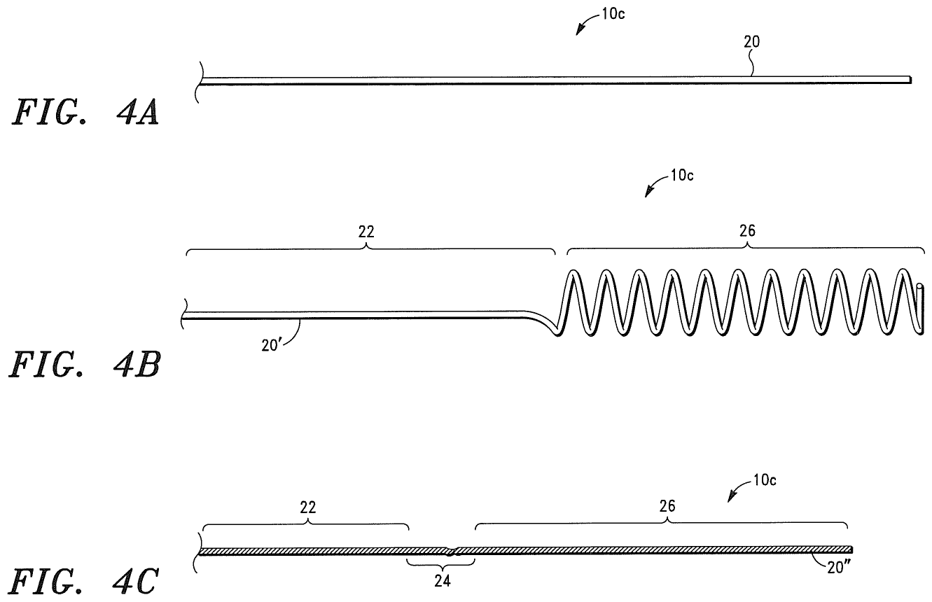

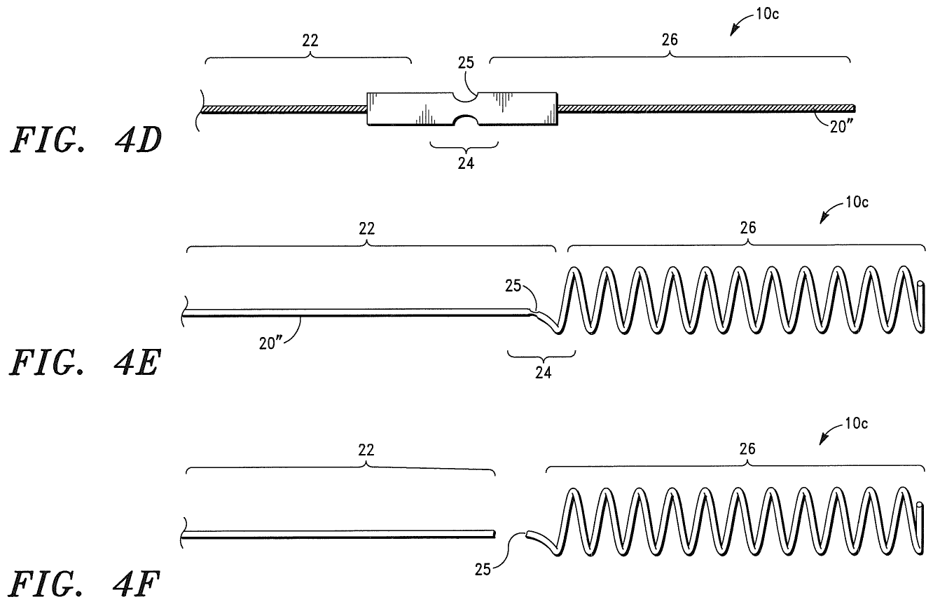

Referring now to FIGS. 4A-4F, there is shown one embodiment of a hybrid endovascular prosthesis of the invention.

As illustrated in FIG. 4A, in a preferred embodiment of the invention, the hybrid endovascular prosthesis 10c similarly comprises a single linear wire stent structure 20.

In a preferred embodiment, the wire stent structure 20 similarly comprises a Ni-Ti alloy, more preferably, Nitinol®.

As illustrated in FIG. 4D and 4E, and discussed in detail below, in a preferred embodiment, after selective differential heat treatments and mechanical processing, the Nitinol® wire stent structure 20 comprises multiple physical property regions: (i) a superelastic region 26 disposed on the distal end of the stent structure, which, as discussed in detail below, is similarly adapted to transition from a pre-deployment substantially linear configuration to a post-deployment coil configuration when subjected to a pre-defined temperature, i.e., A_{f}, (ii) a plastically deformable proximal region or end 22 and (iii) a mechanically deformed, i.e., work hardened, high stress retention region 24 therebetween.

According to the invention, after initial standard Nitinol® processing, e.g., thermal treatment, wherein the Nitinol® wire stent structure 20 comprises a martensite crystal structure, the superelastic region 26 is heated to a temperature of approximately 400° C. to 500° C., wherein the superelastic region 26 exhibits an austinite crystal structure, and the parent coil configuration is formed and fixed (i.e., a Nitinol® shape setting procedure), as shown in FIG. 4B. Thereafter, the Nitinol® wire stent structure, now denoted 20′, is cooled, wherein the Nitinol® wire stent structure 20′ reverts back to the martensite crystal structure and exhibits superelasticity (or pseudoelasticity).

After the Nitinol® wire stent structure 20 is subjected to the aforedescribed Nitinol® shape setting procedure, the Nitinol® wire stent structure 20′ is straightened back to the linear configuration, as shown in FIGS. 4C and 4D, and the plastically deformable proximal region or end 22 is subjected to a further thermal treatment, i.e., annealing, to destroy the superelastic properties and form plastically deformable properties, wherein Nitinol® wire stent structure 20″ is formed. In a preferred embodiment, the thermal treatment comprises annealing the plastically deformable proximal region or end 22 to a temperature in the range of approximately 400° C. to 600° C. for a period of time in the range of approximately 5 min to approximately 20 min.

According to the invention, the localized heating of the plastically deformable proximal region or end 22 can be provided by various conventional means, including, without limitation, electrical resistance heating and induction coil heating.

Referring now to FIGS. 4C and 4D, after the plastically deformable proximal region or end 22 is formed, as described above, the high stress retention region 24 between the superelastic region 26 and plastically deformable proximal region or end 22 is formed.

In a preferred embodiment, the plastically deformable proximal region or end 22 is formed by mechanically deforming and, thereby, work hardening the high stress retention region 24 below the martensite-austenite transformation temperature (A_{f}). Preferably, the high stress region 24 is mechanically deformed in the range of at least 20% to 40%.

According to the invention, the mechanical deformation can result in various fracture zone shapes.

As illustrated in FIG. 4D, in a preferred embodiment, the fracture zone 25 comprises a curved detent configuration, which is preferably formed via a cold rolling process.

Referring now to FIGS. 4E and 4F, according to the invention, when the hybrid endovascular prosthesis 10c shown in FIGS. 4C and 4D is placed in an anatomical passageway, such as a blood vessel, and subjected to a temperature above the martensite-austenite transformation temperature (A_{f}), the superelastic region 26 of Nitinol® wire stent structure 20″ transitions to an austinite crystal structure and reverts back to the parent coil configuration.

A seminal feature of the hybrid endovascular prosthesis 10c is that, according to the invention, during the noted crystal structure transition and reversion back to the parent coil configuration, the high stress region 24 fractures and the superelastic region 26 is severed from plastically deformable proximal region or end 22, as shown in FIG. 4F.

Thus, the need for a conventional delivery catheter is eliminated. Further, and most importantly, the hybrid endovascular prosthesis 10c can readily translate through anatomical passageways with lumen diameters less than 10 um.

As indicated above, in a preferred embodiment, the Nitinol® wire stent structure 20″ and, hence, hybrid endovascular prosthesis formed therewith, further comprises an outer coating.

In some embodiments of the invention, the outer coating comprises one of the aforementioned immunomodulating compounds.

In some embodiments, the outer coating comprises one of the aforementioned ECM compositions.

In some embodiments, the outer coating comprises one of the aforementioned polymeric compositions.

In a preferred embodiment of the invention, the outer coating comprises a thermal insulating outer coating that delays transition of the Nitinol® wire stent structure 20″ an austinite structure and reversion back to the parent coil configuration.

According to the invention, various thermal insulation materials can be employed to form thermal insulating compositions that provide acceptable thermal insulation to delay transition of the Nitinol® wire stent structure 20″ to an austinite structure and reversion back to the parent coil configuration.

In some embodiments of the invention, the thermal insulating composition comprises a biodegradable thermal insulating composition comprising at least one biodegradable thermal insulating material.

According to the invention, suitable thermal insulating materials can comprise polymers having a thermal conductivity less than 0.25 W/(m*K); particularly, polylactide (PLA).

In a preferred embodiment, the thermal insulating outer coating comprises one of the aforementioned ECM-mimicking compositions or ECM/ECM-mimicking compositions comprising PGS.

According to the invention, the ECM-mimicking compositions and ECM/ECM-mimicking compositions can further comprise one or more polymers having a thermal conductivity less than 0.25 W/(m*K), such as PLA.

According to the invention, in addition to providing acceptable thermal insulation to delay transition of the Nitinol® wire stent structure 20″ to an austinite structure and reversion back to the parent coil configuration, the aforementioned ECM-mimicking compositions and ECM/ECM-mimicking compositions will induce remodeling of damaged cardiovascular in an anatomical passageway, e.g., blood vessel, when disposed proximate the internal wall thereof.

According to the invention, the hybrid endovascular prosthesis 10c can similarly further comprise an outer covering or graft, such as endovascular prosthesis 10b shown in FIGS. 3A and 3B.

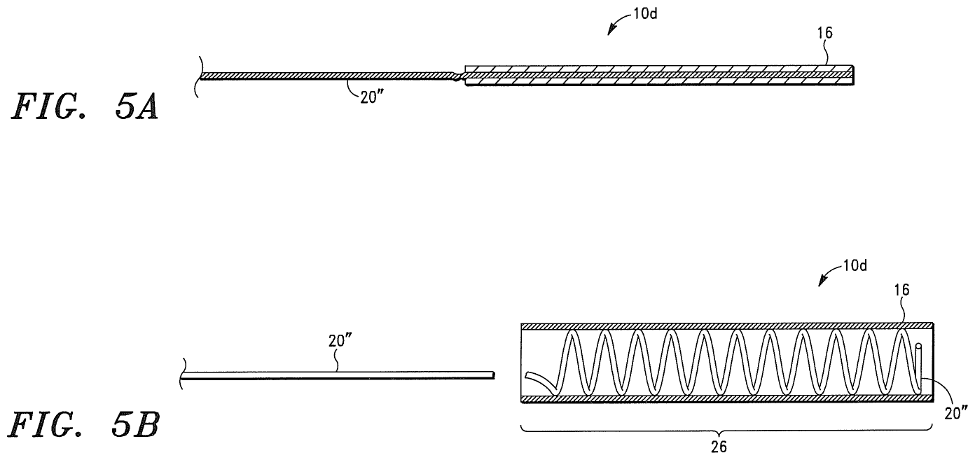

Referring now to FIGS. 5A and 5B, there is shown such an embodiment of a hybrid endovascular prosthesis of the invention, denoted 10d.

As illustrated in FIG. 5A and 5B, in a preferred embodiment, the endovascular prosthesis 10d similarly comprises the same Nitinol® wire stent structure 20″ shown in FIG. 4C and 4D. The endovascular prosthesis 10d further comprises an outer covering or graft 16 that is disposed over the superelastic region 26.

In a preferred embodiment of the invention, the graft 16 is also adapted to transition from a pre-deployment configuration to a post-deployment configuration, as shown in FIG. 5B.

According to the invention, the graft 16 can similarly comprise various biocompatible materials, including, without limitation, one of the aforementioned polymeric and ECM/ECM-mimicking compositions, and an immune privileged collagenous mammalian tissue.

In a preferred embodiment of the invention, the graft 16 similarly comprises one of the aforementioned ECM compositions.

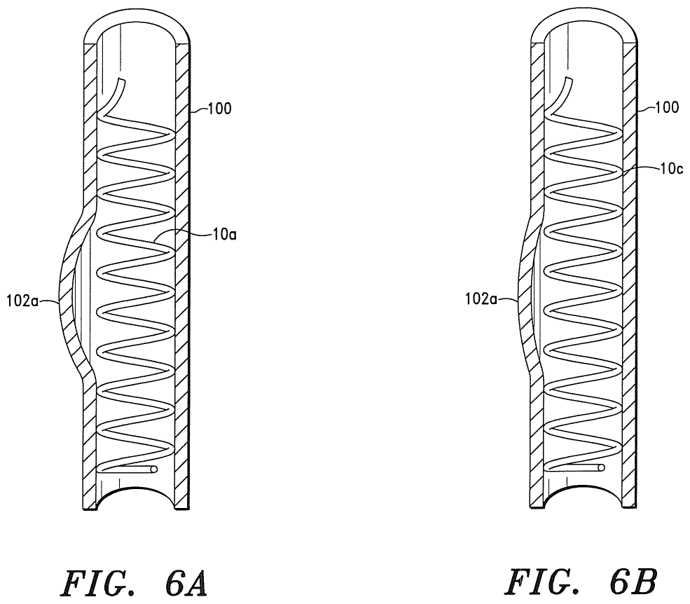

Referring now to FIGS. 6A and 6B, there are shown endovascular prostheses 10a and 10c, respectively, disposed in a blood vessel 100 proximate aneurysm 102a, in accordance with the invention.

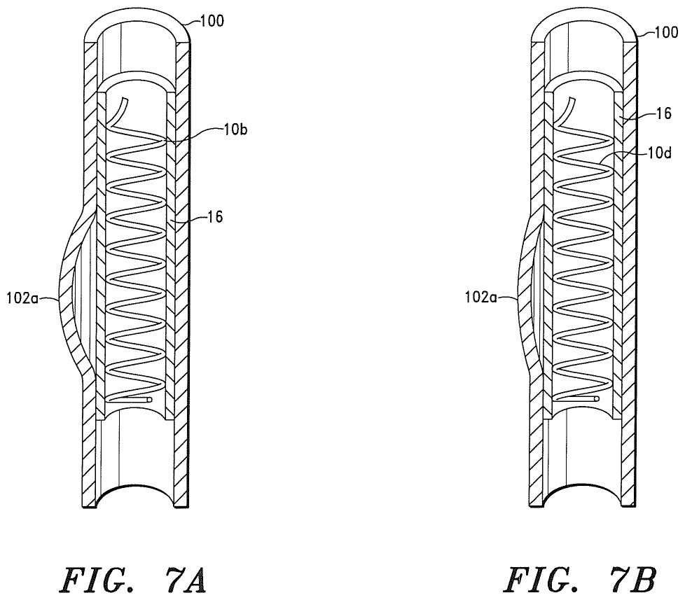

Referring now to FIGS. 7A and 7B, there are shown endovascular prosthesis 10b (FIG. 7A) and the superelastic region 26 of endovascular prosthesis 10d disposed in a blood vessel 100 proximate aneurysm 102a, in accordance with the invention.

As indicated above, in some embodiments of the invention, it is contemplated that, following placement of endovascular prosthesis 10b and the superelastic region 26 of endovascular prosthesis 10d in blood vessel 100 and, hence, tissue associated therewith, endovascular prosthesis 10b and the superelastic region 26 of endovascular prosthesis 10d will induce “modulated healing” of the blood vessel 100 and tissue associated therewith.

As will readily be appreciated by one having ordinary skill in the art, the present invention provides numerous advantages compared to prior art endovascular prostheses. Among the advantages are the following:
The provision of endoluminal prostheses with a pre-deployment diameter less than 10 μm that provide physiologically acceptable radial or hoop strength and longitudinal flexibility;
The provision of endoluminal prostheses that induce remodeling of damaged cardiovascular tissue and regeneration of new cardiovascular tissue when disposed proximate the damaged tissue; and
The provision of endoluminal prostheses that have the capacity to deliver biologically active agents, such as growth factors, and pharmacological agents, such as anti-inflammatories, to cardiovascular tissue, when disposed proximate thereto.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. An endovascular prosthesis, comprising:
a single linear wire stent structure, said wire stent structure comprising a nickel-titanium (Ni-Ti) alloy,
said wire stent structure further comprising a superelastic structure, wherein said wire stent structure is adapted to transition from a pre-deployment configuration, wherein said stent structure can be disposed in a delivery catheter, to an expanded post-deployment coil configuration when said wire stent structure is subjected to a pre-defined temperature,
said wire stent structure further comprising an outer coating, said outer coating comprising an extracellular matrix (ECM) composition comprising acellular ECM derived from a mammalian tissue source.

2. The endovascular prosthesis of claim 1, wherein said mammalian tissue source is selected from the group consisting of small intestine submucosa (SIS), urinary bladder submucosa (UBS), urinary basement membrane (UBM), liver basement membrane (LBM), stomach submucosa (SS), mesothelial tissue, placental tissue, and cardiac tissue.

3. The endovascular prosthesis of claim 1, wherein said ECM composition further comprises at least one exogenously added biologically active agent.

4. The endovascular prosthesis of claim 3, wherein said biologically active agent comprises a cell selected from the group consisting of a human embryonic stem cell, fetal cardiomyocyte, myofibroblast, and mesenchymal stern cell.

5. The endovascular prosthesis of claim 3, wherein said biologically active agent comprises a growth factor selected from the group consisting of a transforming growth factor-alpha (TGF-α), transforming growth factor-beta (TGF-β), fibroblast growth factor-2 (FGF-2), and vascular endothelial growth factor (VEGF).

6. The endovascular prosthesis of claim 1, wherein said ECM composition further comprises a pharmacological agent.

7. The endovascular prosthesis of claim 6, wherein said pharmacological agent comprises an agent selected from the group consisting of an antibiotic, anti-viral agent, analgesic, anti-inflammatory, anti-neoplastic, anti-spasmodic, and anticoagulant and antithrombotic agent.
